(19) United States
(12) Patent Application Publication
(10) Pub. No.: US 2025/0269345 A1
(43) Pub. Date: Aug. 28, 2025

(54) METHODS AND SYSTEMS FOR MOLECULAR DYNAMICS SIMULATION OF COMPLEX AMORPHOUS POLYMERS

(71) Applicant: Ares Materials Inc., Dallas, TX (US)

(72) Inventors: Victor Hugo González-Carbajal, Zapopan (MX); Barbara González-Álvarez, Atotonilco el Alto (MX); Erik Gutierrez-Valladares, Zapopan (MX); Angel Fernando Cisneros-Gaytan, Zapopan (MX); David Arreaga, Garland, TX (US); Adrian Avendano-Bolivar, Prosper, TX (US)

(21) Appl. No.: 18/586,619

(22) Filed: Feb. 26, 2024

(51) Int. Cl. B01J 19/00; G05B 13/02

(52) U.S. Cl. CPC B01J 19/0033; G05B 13/027; B01J 2219/00243

(57) ABSTRACT

A method for simulating a complex amorphous polymer uses a reaction module and a processor. The method includes the processor receiving user input including characteristics of monomers. The processor, via a first trained machine learning model, produces a predicted molecular quantity for each monomer, where the quantities are based on characteristics of the monomers. The reaction module generates predicted representations of the complex amorphous polymer based on the characteristics. The reaction module generates forcefield values for molecules identified in each of the representations. The reaction module analyzes a charge distribution of each of the predicted representations, where identified surplus charges are redistributed among atoms of the representations. The reaction module assembles a simulation box including a reaction product model including the predicted representations, where a quantity of the predicted representations in the simulation box is determined by weights of the representations calculated by a second trained machine learning model.

### FIELD OF THE INVENTION

The present invention relates generally to simulation methods and systems, and more particularly to the simulation of the molecular dynamics of complex amorphous polymers.

### BACKGROUND

Molecular dynamics simulations are widely used in materials design to study the behavior of complex amorphous polymers. These simulations involve modeling the movement and interactions of individual atoms and molecules over time, providing insights into the structure, dynamics, and properties of the materials. Amorphous polymers, in particular, are of great interest due to their diverse applications in various industries, including packaging, electronics, and biomedical fields.

In current academic research, the predominant method for representing polymers involves generating a single repeating unit of arbitrary size and replicating it within the simulation box to predict material properties. This approach has been effective for linear amorphous polymers and is complemented by established techniques for simulating monomer reactions using molecular dynamics and density functional theory.

Current technologies for simulating complex amorphous polymers, although available, exhibit notable limitations and drawbacks. One significant challenge pertains to the representation of non-integer ratios common in amorphous polymers, which can compromise the accuracy and realism of generated polymer structures. Additionally, the stochastic nature of real-world reactions is not consistently captured, potentially affecting the reliability of simulation outcomes. Furthermore, the existing methods may fall short in accurately representing the diversity and true characteristics of simulated systems, thus limiting their applicability to complex polymer systems. The process of creating forcefield files for individual molecules is often time-consuming and manual, potentially impeding the efficiency of simulations.

Moreover, when applied to crosslinked amorphous polymers, the current approach of replicating single repeating units within simulation boxes oversimplifies their complex structural and dynamic properties. Capturing crosslinking effects and addressing non-integer ratios remains challenging within this framework. In addition to these shortcomings, the computational demands of existing methods can restrict the scale and complexity of systems that can be effectively studied. Collectively, these limitations highlight the pressing need for specialized technologies tailored to the unique characteristics and challenges presented by crosslinked amorphous polymers, ultimately enhancing the accuracy and applicability of molecular dynamics simulations in this domain.

In summary, the existing technology for molecular dynamics simulations of complex amorphous polymers provides valuable insights into material properties, but there are still challenges in accurately representing experimental ratios, capturing the stochastic nature of reactions, ensuring true diversity in simulation boxes, and streamlining the creation of forcefield files.

### BRIEF DESCRIPTION OF THE DRAWINGS

For a more complete understanding of the features and advantages of the present disclosure, reference is now made to the detailed description along with the accompanying figures in which corresponding numerals in the different figures refer to corresponding parts and in which:

FIG. 1 is an illustration of a computing machine and a system applications module, in accordance with certain example embodiments;

FIG. 2 is an illustration of a computing system for simulating a complex amorphous polymer in accordance with certain embodiments of the present disclosure;

FIG. 3A is an illustration of molecular diagrams of first and second monomers of a simulation reaction in accordance with certain embodiments of the present disclosure;

FIG. 3B is an illustration of a molecular diagram of a singular reaction product model of a simulation reaction in accordance with certain embodiments of the present disclosure;

FIG. 3C is an illustration of multiple molecular diagrams of possible singular reaction product models of a simulation reaction in accordance with certain embodiments of the present disclosure;

FIG. 4 is an illustration of an output of a reaction module in chart form and depicting descriptors of a plurality of possible products of a simulation reaction in accordance with certain embodiments of the present disclosure; and

FIG. 5, which is an illustration of a flow diagram depicting a method for simulating a complex amorphous polymer in accordance with certain embodiments of the present disclosure.

The illustrated figures are only exemplary and are not intended to assert or imply any limitation with regard to the environment, architecture, design, or process in which different examples may be implemented.

### DETAILED DESCRIPTION

Presented herein are methods and systems for accurately simulating complex amorphous polymers, where the associated output (structures of complex amorphous polymers) is realistic and representative of real-world systems relative to polymerizations. The term “predicted representation” is used herein to describe at least a partial result (in the form of a model) of a simulated polymeric reaction. The term “simulation box” refers to a digital space configured to generate and visualize digital simulations of one or more molecules or compounds (in this case, polymers). For the purposes of this disclosure, the term “complex” is used herein to refer to properties of a polymer such as, for example, temperature dependency, electrical conductivity, thermal stability, breakdown strength, etc., varying from region to region (chain to chain, etc.) within the polymer. Additionally, it should be clear to those skilled in the art that the term “complex”, in regard to polymers, is not limited to the above-mentioned properties and can refer to additional polymer properties.

Due to the advanced nature of the technology presented, there is a wide range of applications for this technology in fields such as materials science, polymer chemistry, and computational chemistry. For example, in materials science, the ability to accurately simulate complex amorphous polymers is imperative for materials design. Disclosed embodiments enable researchers to generate realistic polymer structures, taking into account non-integer ratios and stochastic reactions. By accurately simulating the behavior of these polymers, valuable insights can be gained into their material properties, such as, for example, mechanical strength, thermal stability, and electrical conductivity. This can aid in the development of new materials with tailored properties for various applications, including in industries such as automotive, aerospace, electronics, and packaging.

In regards to polymer chemistry, the ability to predict and understand the behavior of amorphous polymers is necessary for designing new polymer materials with specific, desired properties. Disclosed embodiments provide to researchers the ability to simulate the reactions and structures of amorphous polymers, facilitating the design of polymers with specific functionalities and/or performance characteristics. This can be applied to technological areas such as drug delivery systems, coatings, adhesives, and biomaterials.

In regards to computational chemistry, the algorithms and modules disclosed herein may offer a valuable tool for analyzing the behavior of complex amorphous polymers at the molecular level. This analyzing can aid in the development of new simulation methodologies and forcefields for polymer systems. Additionally, the clustering algorithm disclosed that is utilized for generating representative structures can be applied to other molecular systems, which can enhance the diversity and accuracy of simulations in various fields of chemistry.

Overall, the disclosed embodiments have significant application prospects in materials science, polymer chemistry, and computational chemistry, while also addressing the demand for accurate and realistic simulations of complex amorphous polymers for materials design. The potential market for this technology spans industries such as, for example, automotive, aerospace, electronics, packaging, pharmaceuticals, and chemical manufacturing.

While the making and using of various embodiments of the present disclosure are discussed in detail below, it should be appreciated that the present disclosure provides many applicable inventive concepts, which can be embodied in a wide variety of specific contexts. The specific embodiments discussed herein are merely illustrative and do not delimit the scope of the present disclosure. In the interest of clarity, not all features of an actual implementation may be described in the present disclosure.

Unless otherwise indicated, all numbers expressing quantities of components, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit.

Presented herein is a computer-implemented method for simulating a complex amorphous polymer using a reaction module and a processor. The method comprises receiving, by the processor, user input representative of characteristics of at least one monomer. The processor, via a first trained machine learning model, produces a predicted molecular quantity for each of the at least one monomer, where each of the predicted molecular quantities are based on the characteristics of the at least one monomer. The reaction module generates a plurality of predicted representations of the complex amorphous polymer based on the characteristics of the at least one monomer. The reaction module generates forcefield values for each of one or more molecules identified in each of the plurality of predicted representations. The reaction module analyzes a charge distribution of each of the plurality of predicted representations, where one or more surplus charges identified in one or more of the plurality of predicted representations are redistributed among one or more atoms of a respective one or more of the plurality of predicted representations. The reaction module assembles a simulation box comprising a reaction product model including the plurality of predicted representations, where a quantity of each of the plurality of predicted representations in the simulation box is determined by weights of each of the plurality of predicted representations calculated by the second trained machine learning model.

Additionally presented herein is a computer program product for simulating a complex amorphous polymer. The computer program product comprises a computer readable storage medium having program instructions embodied therewith, where the program instructions are executable by a processor to cause the processor to perform receiving, by the processor, user input representative of characteristics of at least one monomer. The processor, via a first trained machine learning model, produces a predicted molecular quantity for each of the at least one monomer, where each of the predicted molecular quantities is based on the characteristics of the at least one monomer. A reaction module generates a plurality of predicted representations of the complex amorphous polymer based on the characteristics of the at least one monomer. The reaction module generates forcefield values for each of one or more molecules identified in each of the plurality of predicted representations. The reaction module analyzes a charge distribution of each of the plurality of predicted representations, where one or more surplus charges identified in one or more of the plurality of predicted representations are redistributed among one or more atoms of a respective one or more of the plurality of predicted representations. The reaction module assembles a simulation box comprising a reaction product model including the plurality of predicted representations, where a quantity of each of the plurality of predicted representations in the simulation box is determined by weights of each of the plurality of predicted representations calculated by a second trained machine learning model.

Additionally presented herein is a computing system comprising a processor and a computer-readable storage device coupled to the processor. The computing system further comprises a reaction module coupled to the processor and program instructions stored on the computer readable storage device for execution by the processor via a memory, where execution of the instructions by the processor configures the computing device to perform a complex amorphous polymer simulation method comprising receiving, by the processor, user input representative of characteristics of at least one monomer. The processor, via a first trained machine learning model, produces a predicted molecular quantity for each of the at least one monomer, where each of the predicted molecular quantities is based on the characteristics of the at least one monomer. The reaction module generates a plurality of predicted representations of the complex amorphous polymer based on the characteristics of the at least one monomer. The reaction module generates forcefield values for each of one or more molecules identified in each of the plurality of predicted representations. The reaction module analyzes a charge distribution of each of the plurality of predicted representations, where one or more surplus charges identified in one or more of the plurality of predicted representations are redistributed among one or more atoms of a respective one or more of the plurality of predicted representations. The reaction module assembles a simulation box comprising a reaction product model including the plurality of predicted representations, where a quantity of each of the plurality of predicted representations in the simulation box is determined by weights of each of the plurality of predicted representations calculated by a second trained machine learning model.

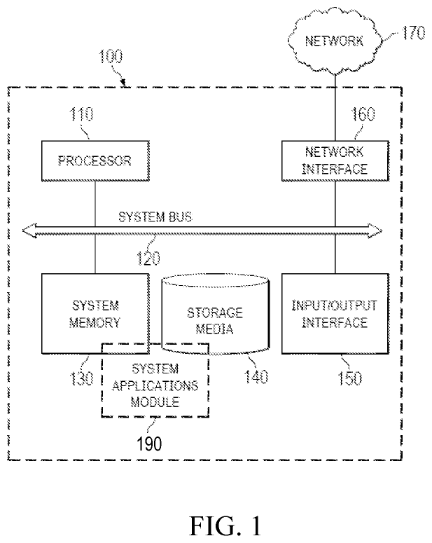

Referring now to FIG. 1, illustrated is a computing machine 100 and a system applications module 190, in accordance with example embodiments. The computing machine 100 can correspond to any of the various computers, mobile devices, laptop computers, Internet of Things (IoT), servers, embedded systems, or computing systems presented herein. The module 190 can comprise one or more hardware or software elements, e.g. other OS application and user and kernel space applications, designed to facilitate the computing machine 100 in performing the various methods and processing functions presented herein. The computing machine 100 can include various internal or attached components such as a processor 110, system bus 120, system memory 130, storage media 140, input/output interface 150, a network interface 160 for communicating with a network 170, e.g. cellular/GPS, Bluetooth, WIFI, or Devicenet, EtherCAT, Analog, RS485, etc.

The computing machines can be implemented as a conventional computer system, an embedded controller, a laptop, a server, a mobile device, a smartphone, a wearable computer, a customized machine, any other hardware platform, or any combination or multiplicity thereof. The computing machines can be a distributed system configured to function using multiple computing machines interconnected via a data network or bus system.

Processor 110 can be designed to execute code instructions in order to perform the operations and functionality described herein, manage request flow and address mappings, and to perform calculations and generate commands. Processor 110 can be configured to monitor and control the operation of the components in the computing machines. Processor 110 can be a general purpose processor, a processor core, a multiprocessor, a reconfigurable processor, a microcontroller, a digital signal processor (“DSP”), an application specific integrated circuit (“ASIC”), a controller, a state machine, gated logic, discrete hardware components, any other processing unit, or any combination or multiplicity thereof. Processor 110 can be a single processing unit, multiple processing units, a single processing core, multiple processing cores, special purpose processing cores, co-processors, or any combination thereof. According to certain embodiments, processor 110 along with other components of computing machine 100 can be a software based or hardware based virtualized computing machine executing within one or more other computing machines. In another embodiment, for example, such aforementioned components can be implemented as software installed and stored in a persistent storage device, which can be loaded and executed in a memory by processor 110 to carry out the processes or operations described throughout this application. Alternatively, the components can be implemented as executable code programmed or embedded into dedicated hardware such as an integrated circuit (e.g., an application specific IC or ASIC), a digital signal processor (DSP), or a field programmable gate array (FPGA), which can be accessed via a corresponding driver and/or operating system from an application. Furthermore, the components can be implemented as specific hardware logic in a processor or processor core as part of an instruction set accessible by a software component via one or more specific instructions.

The system memory 130 can include non-volatile memories such as read-only memory (“ROM”), programmable read-only memory (“PROM”), erasable programmable read-only memory (“EPROM”), flash memory, or any other device capable of storing program instructions or data with or without applied power. The system memory 130 can also include volatile memories such as random access memory (“RAM”), static random access memory (“SRAM”), dynamic random access memory (“DRAM”), and synchronous dynamic random access memory (“SDRAM”). Other types of RAM also can be used to implement the system memory 130. The system memory 130 can be implemented using a single memory module or multiple memory modules. While the system memory 130 is depicted as being part of the computing machine, one skilled in the art will recognize that the system memory 130 can be separate from the computing machine 100 without departing from the scope of the subject technology. It should also be appreciated that the system memory 130 can include, or operate in conjunction with, a non-volatile storage device such as the storage media 140.

The storage media 140 can include a hard disk, a floppy disk, a compact disc read-only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 140 can store one or more operating systems, application programs and program modules, data, or any other information. The storage media 140 can be part of, or connected to, the computing machine. The storage media 140 can also be part of one or more other computing machines that are in communication with the computing machine such as servers, database servers, cloud storage, network attached storage, and so forth.

The applications module 190 and other OS application modules can comprise one or more hardware or software elements configured to facilitate the computing machine with performing the various methods and processing functions presented herein. The applications module 190 and other OS application modules can include one or more algorithms or sequences of instructions stored as software or firmware in association with the system memory 130, the storage media 140 or both. The storage media 140 can therefore represent examples of machine or computer readable media on which instructions or code can be stored for execution by the processor 110. Machine or computer readable media can generally refer to any medium or media used to provide instructions to the processor 110. Such machine or computer readable media associated with the applications module 190 and other OS application modules can comprise a computer software product. It should be appreciated that a computer software product comprising the applications module 190 and other OS application modules can also be associated with one or more processes or methods for delivering the applications module 190 and other OS application modules to the computing machine via a network, any signal-bearing medium, or any other communication or delivery technology. The applications module 190 and other OS application modules can also comprise hardware circuits or information for configuring hardware circuits such as microcode or configuration information for an FPGA or other PLD. In one exemplary embodiment, applications module 190 and other OS application modules can include algorithms capable of performing the functional operations described by the flow charts (modes of operation) computer systems presented herein.

The input/output (“I/O”) interface 150 can be configured to couple to one or more external devices, to receive data from the one or more external devices, and to send data to the one or more external devices. Such external devices along with the various internal devices can also be known as peripheral devices. The I/O interface 150 can include both electrical and physical connections for coupling the various peripheral devices to the computing machine or the processor 110. The I/O interface 150 can be configured to communicate data, addresses, and control signals between the peripheral devices, the computing machine, or the processor 110. The I/O interface 150 can be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 150 can be configured to implement only one interface or bus technology. Alternatively, the I/O interface 150 can be configured to implement multiple interfaces or bus technologies. The I/O interface 150 can be configured as part of, all of, or to operate in conjunction with, the system bus 120. The I/O interface 150 can include one or more buffers for buffering transmissions between one or more external devices, internal devices, the computing machine, or the processor 120.

The I/O interface 120 can couple the computing machine to various input devices including mice, touch-screens, scanners, electronic digitizers, sensors, receivers, touchpads, trackballs, cameras, microphones, keyboards, any other pointing devices, or any combinations thereof. The I/O interface 120 can couple the computing machine to various output devices including video displays, speakers, printers, projectors, tactile feedback devices, automation control, robotic components, actuators, motors, fans, solenoids, valves, pumps, transmitters, signal emitters, lights, and so forth.

The computing machine 100 can operate in a networked environment using logical connections through the NIC 160 to one or more other systems or computing machines across a network. The network can include wide area networks (WAN), local area networks (LAN), intranets, the Internet, wireless access networks, wired networks, mobile networks, telephone networks, optical networks, or combinations thereof. The network can be packet switched, circuit switched, of any topology, and can use any communication protocol. Communication links within the network can involve various digital or an analog communication media such as fiber optic cables, free-space optics, waveguides, electrical conductors, wireless links, antennas, radiofrequency communications, and so forth.

The processor 110 can be connected to the other elements of the computing machine or the various peripherals discussed herein through the system bus 120. It should be appreciated that the system bus 120 can be within the processor 110, outside the processor 110, or both. According to some embodiments, any of the processors 110, the other elements of the computing machine, or the various peripherals discussed herein can be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions unless otherwise disclosed for an exemplary embodiment. Further, a skilled programmer would be able to write such a computer program to implement an embodiment of the disclosed embodiments based on the appended flow charts, algorithms and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

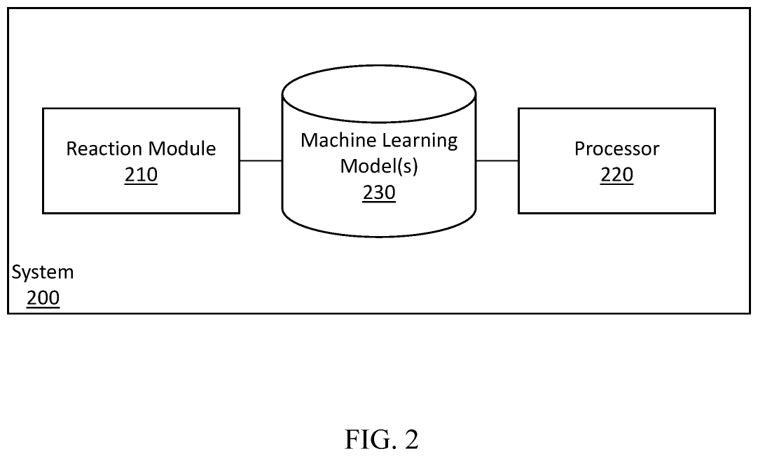

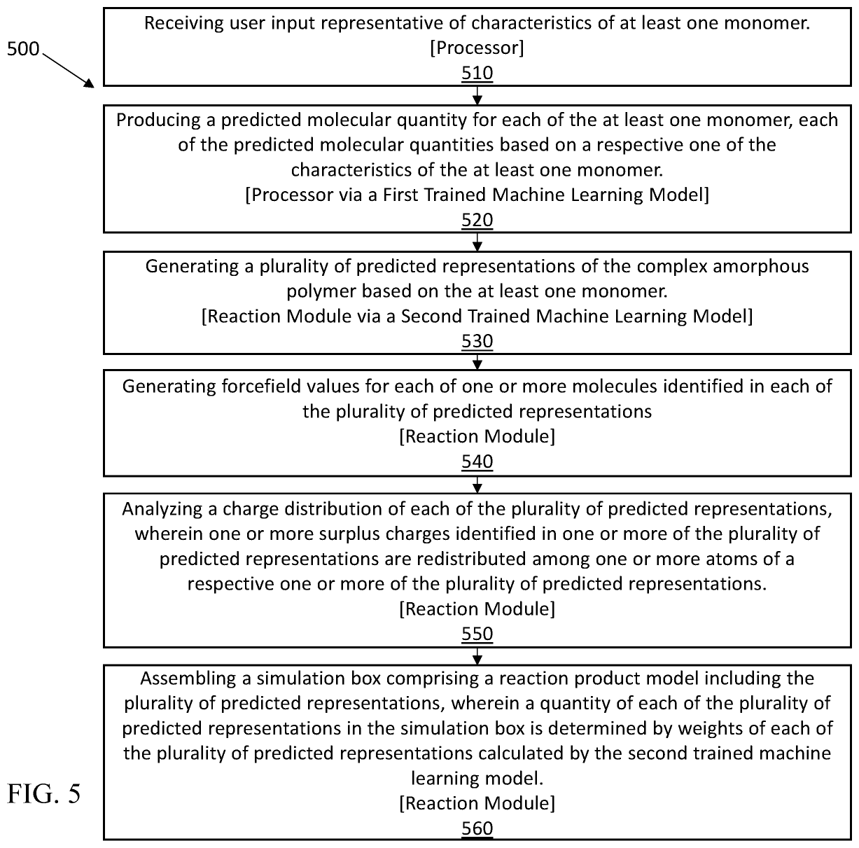

FIG. 2 is an illustration of a computing system 200 for simulating a complex amorphous polymer in accordance with certain embodiments of the present disclosure. As shown, system 200 includes a reaction module 210, a processor 220, and one or more machine learning models 230. Examples of models 230, in embodiments, include a deep neural network, a decision tree algorithm, or a clustering algorithm. Reaction module 210 and processor 220 are adapted to facilitate training of, and configure, machine learning models 230 by performing the process presented in FIG. 5. For example, reaction module 210 enables the following: the generating of a plurality of predicted representations of a complex amorphous polymer, the generating of forcefield values for each of one or more molecules identified in each of the plurality of predicted representations, the analyzing of a charge distribution of each of the plurality of predicted representations, and the assembling of a simulation box comprising a reaction product model including the plurality of predicted representations. As a further example, processor 220 enables the receiving of user input representative of characteristics of at least one monomer and (via a first trained machine learning model) the producing of a predicted molecular quantity for each of the at least one monomer.

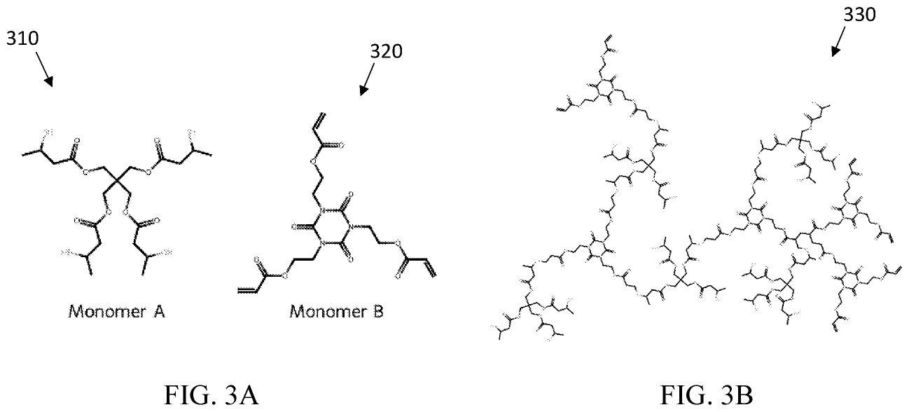

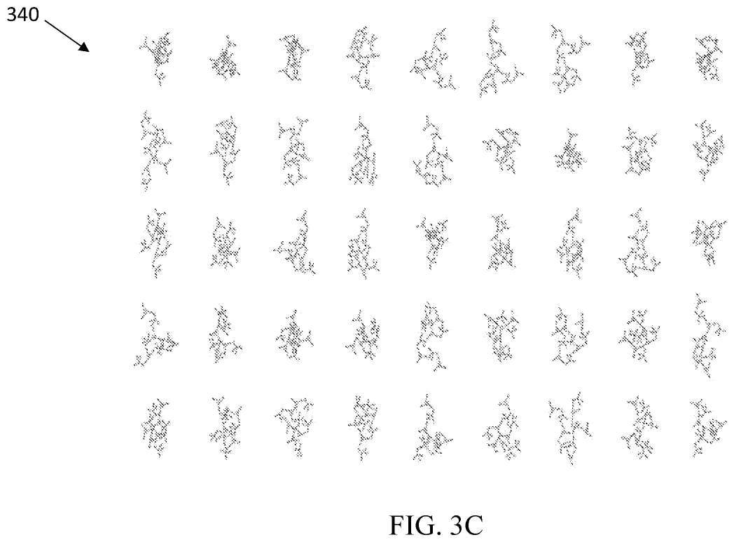

Program instructions stored on the computer readable storage device are configured for execution by the processor via a memory (similar to the system memory 130 of FIG. 1) coupled to the processor (for example, processor 220). The instructions are configured to render computing system 200 capable of performing a number of operations in a computer-implemented method for simulating a complex amorphous polymer (presented similarly in FIG. 5). The method includes receiving, by the processor 220, user input representative of characteristics of at least one monomer (for example, Monomer A and Monomer B of molecular diagrams 310,320 of FIG. 3A). The processor 220, via a first trained machine learning model (of machine learning model(s) 230), then produces a predicted molecular quantity for each of the at least one monomer, where each of the predicted molecular quantities are based on the characteristics of the at least one monomer. Once the processor 220, via the first trained machine learning model, produces the predicted molecular quantities, the reaction module 210 then generates a plurality of predicted representations (depicted as singular reaction product models 340 of FIG. 3C) of the complex amorphous polymer based on the characteristics of the at least one monomer.

The reaction module 210 then generates forcefield values for each of one or more molecules identified in each of the plurality of predicted representations. The reaction module 210 then analyzes a charge distribution of each of the plurality of predicted representations, where one or more surplus charges identified in one or more of the plurality of predicted representations are redistributed among one or more atoms of a respective one or more of the plurality of predicted representations. The reaction module 210 then assembles a simulation box comprising a reaction product model including the plurality of predicted representations, where a quantity of each of the plurality of predicted representations in the simulation box is determined by weights of each of the plurality of predicted representations calculated by a second trained machine learning model (of machine learning model(s) 230).

In one embodiment, the characteristics of the at least one monomer comprise at least one of: a simplified molecular-input line-entry system (SMILES) representation or a desired stoichiometric ratio relative to the singular reaction product models 340 (see FIG. 3C).

In a further embodiment, execution of the instructions by the processor configures computing system 200 to additionally perform calculating, by the reaction module 210, a molecular topological descriptor for each of the plurality of predicted representations.

In a further embodiment, execution of the instructions by the processor configures computing system to additionally perform clustering, by the reaction module 210 via the second trained machine learning model, each of the plurality of predicted representations into one or more groups, where each of the one or more groups are based on one or more structural similarities of each of the plurality of predicted representations.

In a further embodiment, execution of the instructions by the processor configures computing system to additionally perform generating, by the reaction module 210, geometric values for each of the plurality of predicted representations.

In a further embodiment, each of the first trained machine learning model and the second trained machine learning model are selected from the group consisting of: a deep neural network, a decision tree algorithm, or a clustering algorithm.

In a further embodiment, each of the plurality of predicted representations comprises a simplified molecular-input line-entry system (SMILES) representation of the complex amorphous polymer.

According to an embodiment, a computer program product for simulating a complex amorphous polymer is provided. The computer program product includes a computer readable storage device embodying program instructions executable by a processor to cause the processor to perform a plurality of steps. These steps may correlate to any process steps/functions relative to FIG. 5.

FIG. 3A is an illustration of molecular diagrams of first and second monomers 310,320 of a simulation reaction in accordance with certain embodiments of the present disclosure. As shown, first and second monomer diagrams 310,320 are presented via input/output interface 150 of computing machine 100. Diagrams 310,320 are produced via the processor 220 receiving user input representative of characteristics of at least one monomer (in this case, two monomers). In this case, the characteristics include a simplified molecular-input line-entry system (SMILES) representation and a desired stoichiometric ratio relative to the singular reaction product model 330 (see FIG. 3B). For Monomer A of diagram 310, exemplary characteristics include the SMILES representation of Monomer A:

CC(CC(═O)OCC(COC(═O)CC(C)S)(COC(═O)CC(C)S)COC(═O)CC(C)S)S

and a desired stoichiometric ratio of six. For Monomer B of diagram 320, exemplary characteristics include the SMILES representation of Monomer B:

C═CC(═O)OCCN₁C(═O)N(C(═O)N(C₁═O)CCOC(═O)C═C)CCOC(═O)C═C

and a desired stoichiometric ratio of six. In additional embodiments, characteristics may further include functional group information of a monomer (for example, thiol functional groups of Monomer A and alkene functional groups of Monomer B). Once the processor 220 receives user input representative of characteristics of Monomers A and B, processor 220 produces, via a first trained machine learning model, a predicted molecular quantity for each of the monomers, where each of the predicted molecular quantities are based on the characteristics of the monomers. In embodiments, the first trained machine learning model embodies rules including: optimizing chain lengths via the inclusion of non-integer ratios (commonly found in amorphous polymers) in order to maintain experimental stoichiometric ratios of monomers within user-defined size limits (in relation to the predicted molecular quantities) and in order to optimize chain lengths of predicted representations (such as, for example, singular reaction product models 340 of FIG. 3C) within user-defined size limits. By virtue of this feature, the predicted representations of the polymers accurately imitate their real-world counterparts, allowing for more precise materials design.

FIG. 3B is an illustration of a molecular diagram of a singular reaction product model 330 of a simulation reaction in accordance with certain embodiments of the present disclosure. As shown, singular reaction product model 330, as well as the singular reaction product model's SMILES representation, are produced via reaction module 210. Reaction module 210 receives, as input, the characteristics of Monomers A and B (the simplified molecular-input line-entry system (SMILES) representations and the desired stoichiometric ratios relative to the singular reaction product model 330 of Monomers A and B) and is configured to replicate the stochastic nature of real-world polymeric reactions in order to generate a SMILES representation of one of a possible plurality of reacted polymers according to the probability of each reaction in the polymerization of Monomers A and B. In order to govern the reactions in the polymerization, reaction module 210 is configured to derive probabilities from kinetic constants relative to the reactions. In relation to polymerization reactions, reaction module 210 is configured to be versatile and can handle various types of reactions such as, for example, thiol-ene homopolymerization or acrylate homopolymerization. It is noted that the utilization of non-integer stoichiometric ratios as well as the replication of the stochastic nature of real-world polymeric reactions by reaction module 210 provides adds to the reliability of the SMILES representations/simulations of the plurality of reacted polymers. Furthermore, the probabilistic approach of reaction module 210 to execute reactions (as opposed to the reliance on molecular simulations to execute reactions) results in an increase in time and efficiency in regards to reaction execution.

As shown in FIG. 3C, multiple molecular diagrams of possible/predicted singular reaction product models 340 (as well as the singular reaction product models' SMILES representations) of the simulated polymerization reaction of Monomers A and B are illustrated. These possible singular reaction product models 340 (representing predicted representations of the polymerization reaction) are also produced by reaction module 210 using the parameters presented above. It is noted that the models 340 in FIG. 3C are exemplary and may not include the entirety of the possible/predicted singular reaction product models 340 in relation to the polymerization reaction of Monomers A and B. In an embodiment, in order to generate the SMILES representations, reaction module 210 is configured to implement as software such as, for example RDKit (an open-source cheminformatics software).

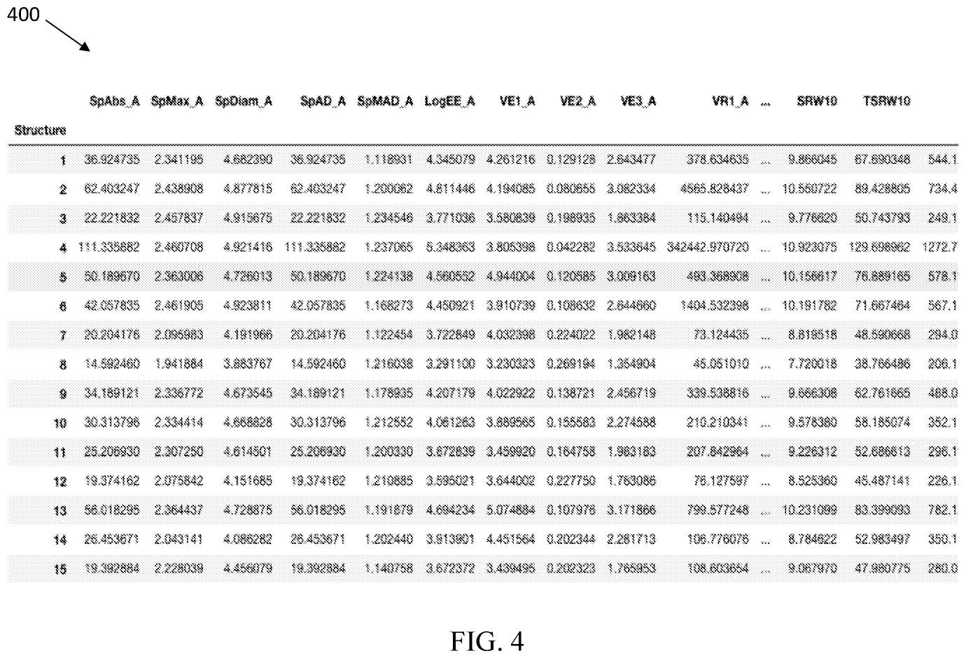

FIG. 4 is an illustration of an output 400 of a reaction module 210 in chart form and depicting descriptors of a plurality of possible products of a simulation reaction in accordance with certain embodiments of the present disclosure. As shown, a subset of an entire set of molecular topological descriptors is presented in the chart shown. Reaction module 210 calculates molecular topological descriptors for each of the possible/predicted singular reaction product models 340 (representing predicted representations of the polymerization reaction) generated in order to quantitatively assess the structural characteristics of the reaction product models 340. Once the topological descriptors are calculated, reaction module 210 via a second trained machine learning model (a clustering algorithm/machine learning model) clusters each of the plurality of predicted representations into one or more groups, where each of the one or more groups are based on one or more descriptor/structural similarities of each of the plurality of predicted representations (SMILES representations). This process generates a subset of predicted representations of the complex amorphous polymer that represent an entire range of possible outcomes (predicted representations that can be considered the most representative of the products of the polymerization reaction), while also keeping the number of singular reaction product models 340 within a manageable limit for computer simulation. This, in turn, may capture the diverse range of polymer conformations and structural variations that can result from the permissible reactions. In embodiments, the second trained machine learning model/clustering algorithm facilitates the automatic identification of the predicted representations without introducing predefined biases (only based on structural similarities), which results in a list of predicted representations that ensures an accurate reflection of the real-world diversity of the polymer across the entire space of possible reactions.

Once the clustering process has been carried out, reaction module 210 may generate geometric values for each of the plurality of predicted representations that are derived from the SMILES representations. The generation of geometric values may include assigning three-dimensional coordinates to the predicted representations and subsequently conducting a geometric optimization. For the purposes of this disclosure, it is understood that geometry optimization refers to a quantum mechanical calculation for predicting a salient shape of a molecule. Reaction module 210 may also generate forcefield values for each molecule identified in each predicted representation (in the form of a computer file), where the forcefield values are utilized by molecular dynamics software to facilitate accurate predicted representations. Also, by virtue of this feature, the predicted representations are ensured to interact realistically with their environment, leading to valuable insights into the behaviors and properties of the polymers.

Reaction module 210 may subsequently analyze a charge distribution of each of the predicted representations, where one or more surplus charges identified in each of the predicted representations are redistributed among one or more atoms of each of the predicted representations in a weighted fashion. This analysis ensures that a neutral charge is maintained in each of the predicted representations. In embodiments, any suitable charge distribution algorithm may be utilized in order to carry out the charge distribution analysis. For example, the algorithm for analyzing the charge distribution may be configured to analyze one or more of: continuous charge distribution, linear charge distribution, surface charge distribution, volume charge distribution, etc. After the charge distribution step is carried out, reaction module 210 assembles a simulation box comprising a reaction product model including the plurality of predicted representations, where a quantity of each of the plurality of predicted representations in the simulation box is determined by weights of each of the plurality of predicted representations calculated by the second trained machine learning model (clustering algorithm).

### Example Process

Reference is now made to FIG. 5, which illustrates a flow diagram depicting a method 500 for simulating a complex amorphous polymer in accordance with certain embodiments of the present disclosure. Flow diagram of method 500 is illustrated as a process in logical flow diagram format, wherein the flow diagram represents a sequence of operations that can be implemented in hardware, software, or a combination thereof. In the context of software, the process represents computer-executable instructions that, when executed by one or more processors, perform the recited operations. Generally, computer-executable instructions may include routines, programs, objects, components, data structures, and the like that perform functions or implement abstract data types. The order in which the operations are described is not intended to be construed as a limitation, and any number of the described processes can be combined in any order and/or performed in parallel to implement the process. For discussion purposes, the method 500 is described with reference to the architecture of an environment 100, a computing system 200 of FIG. 2, and molecular diagrams/models relative to a simulation reaction of FIGS. 3A-3C.

At block 510, a processor 220 receives user input representative of characteristics of at least one monomer (for example, Monomer A and Monomer B of molecular diagrams 310,320).

At block 520, the processor 220, via a first trained machine learning model (of machine learning model(s) 230), produces a predicted molecular quantity for each of the at least one monomer, where each of the predicted molecular quantities are based on the characteristics of the at least one monomer.

At block 530, a reaction module 210 generates a plurality of predicted representations (such as singular reaction product models 340) of the complex amorphous polymer based on the characteristics of the at least one monomer.

At block 540, the reaction module 210 generates forcefield values for each of one or more molecules identified in each of the plurality of predicted representations.

At block 550, the reaction module 210 analyzes a charge distribution of each of the plurality of predicted representations, where one or more surplus charges identified in one or more of the plurality of predicted representations are redistributed among one or more atoms of a respective one or more of the plurality of predicted representations.

At block 560, the reaction module 210 assembles a simulation box comprising a reaction product model including the plurality of predicted representations, where a quantity of each of the plurality of predicted representations in the simulation box is determined by weights of each of the plurality of predicted representations calculated by the second trained machine learning model.

In one embodiment, the characteristics of the at least one monomer comprise at least one of: a simplified molecular-input line-entry system (SMILES) representation or a desired stoichiometric ratio relative to the reaction product model.

In a further embodiment, the integration workflow of the flowchart of method 500 further includes calculating, by the reaction module 210 via the second trained machine learning model, a molecular topological descriptor for each of the plurality of predicted representations.

In a further embodiment, the integration workflow of the flowchart of method 500 further includes clustering, by the reaction module 210 via the second trained machine learning model, each of the plurality of predicted representations into one or more groups, where each of the one or more groups are based on one or more structural similarities of each of the plurality of predicted representations.

In a further embodiment, the integration workflow of the flowchart of method 500 further includes generating, by the reaction module 210, geometric values for each of the plurality of predicted representations.

In a further embodiment, each of the first trained machine learning model and the second trained machine learning model are selected from the group consisting of: a deep neural network, a decision tree algorithm, or a clustering algorithm.

In a further embodiment, each of the plurality of predicted representations comprises a simplified molecular-input line-entry system (SMILES) representation of the complex amorphous polymer.

For the purposes of this disclosure, the terms “possible/predicted reaction product models”, “predicted reaction product models”, and “predicted representations” may be synonymous.

It is noted that the complex amorphous polymers simulated by the methods and systems presented herein may be utilized in a display/display device/display panel that may be flexible or inflexible. The complex amorphous polymers may be incorporated into one or more layers of the display/display device/display panel that include, but are not limited to: a seal layer, a cathode layer, an emissive layer, an adhesive layer, a conductive layer, an anode layer, and a substrate layer. It is understood that the display/display device/display panel may include additional film layers that are not mentioned previously.

In an embodiment of the present disclosure, an electronic device may be provided that may utilize one or more complex amorphous polymers of the present disclosure in a display/display device/display panel associated with the electronic device. For exemplary purposes, the electronic device may be any of: a smart phone, a mobile phone, a video phone, a camera, a wearable device (such as electronic clothing, an electronic accessory, a smart watch, a head-mounted apparatus, an electronic bracelet, an electronic necklace, or an electronic tattoo), a personal digital assistant (PDA), a desktop computer (PC), a laptop PC, a netbook PC, a portable multimedia player (PMP), a digital audio player, a mobile medical apparatus, an e-book reader, etc. In additional embodiments, electronic device may be a smart home appliance including a display/display device/display panel. For exemplary purposes, the smart home appliance may be any of: an electronic key, a stereo, a TV, a set-top box, a television (TV) box, a video recorder, a game console, a vacuum cleaner, a digital video disk (DVD) player, a refrigerator, an air conditioner, an oven, a dryer, an air purifier, a microwave oven, a washing machine, an electronic dictionary, an electronic photo frame, etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the description herein.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification. Further, the following clauses represent additional embodiments of the disclosure and should be considered within the scope of the disclosure:

Clause 1, a computer-implemented method for simulating a complex amorphous polymer using a reaction module and a processor, the method comprising: receiving, by the processor, user input representative of characteristics of at least one monomer; producing, by the processor via a first trained machine learning model, a predicted molecular quantity for each of the at least one monomer, each of the predicted molecular quantities based on the characteristics of the at least one monomer; generating, by the reaction module, a plurality of predicted representations of the complex amorphous polymer based on the characteristics of the at least one monomer; generating, by the reaction module, forcefield values for each of one or more molecules identified in each of the plurality of predicted representations; analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

Clause 2, the method of Clause 1, wherein the characteristics of the at least one monomer comprise at least one of: a simplified molecular-input line-entry system (SMILES) representation or a desired stoichiometric ratio relative to the reaction product model.

Clause 3, the method of Clause 1, further comprising calculating, by the reaction module, a molecular topological descriptor for each of the plurality of predicted representations.

Clause 4, he method of Clause 1, further comprising clustering, by the reaction module via the second trained machine learning model, each of the plurality of predicted representations into one or more groups, each of the one or more groups based on one or more structural similarities of each of the plurality of predicted representations.

Clause 5, the method of Clause 1, further comprising generating, by the reaction module, geometric values for each of the plurality of predicted representations.

Clause 6, the method of Clause 1, wherein each of the first trained machine learning model and the second trained machine learning model are selected from the group consisting of: a deep neural network, a decision tree algorithm, or a clustering algorithm.

Clause 7, the method of Clause 1, wherein each of the plurality of predicted representations comprises a simplified molecular-input line-entry system (SMILES) representation of the complex amorphous polymer.

Clause 8, a computer program product for simulating a complex amorphous polymer, the computer program product comprising a computer readable storage device having program instructions embodied therewith, the program instructions executable by a processor to cause the processor to perform: receiving, by a processor, user input representative of characteristics of at least one monomer; producing, by the processor via a first trained machine learning model, a predicted molecular quantity for each of the at least one monomer, each of the predicted molecular quantities based on the characteristics of the at least one monomer; generating, by a reaction module, a plurality of predicted representations of the complex amorphous polymer based on the characteristics of the at least one monomer; generating, by the reaction module, forcefield values for each of one or more molecules identified in each of the plurality of predicted representations; analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

Clause 9, the computer program product of Clause 8, wherein the characteristics of the at least one monomer comprise at least one of: a simplified molecular-input line-entry system (SMILES) representation or a desired stoichiometric ratio relative to the reaction product model.

Clause 10, the computer program product of Clause 8, further comprising calculating, by the reaction module, a molecular topological descriptor for each of the plurality of predicted representations.

Clause 11, the computer program product of Clause 8, further comprising clustering, by the reaction module via the second trained machine learning model, each of the plurality of predicted representations into one or more groups, each of the one or more groups based on one or more structural similarities of each of the plurality of predicted representations.

Clause 12, the computer program product of Clause 8, further comprising generating, by the reaction module, geometric values for each of the plurality of predicted representations.

Clause 13, the computer program product of Clause 8, wherein each of the first trained machine learning model and the second trained machine learning model are selected from the group consisting of: a deep neural network, a decision tree algorithm, or a clustering algorithm.

Clause 14, the computer program product of Clause 8, wherein each of the plurality of predicted representations comprises a simplified molecular-input line-entry system (SMILES) representation of the complex amorphous polymer.

Clause 15, a computing system comprising: a processor; a computer-readable storage device coupled to the processor; a reaction module coupled to the processor; and program instructions stored on the computer readable storage device for execution by the processor via a memory, wherein execution of the instructions by the processor configures the computing system to perform a complex amorphous polymer simulation method comprising: receiving, by the processor, user input representative of characteristics of at least one monomer; producing, by the processor via a first trained machine learning model, a predicted molecular quantity for each of the at least one monomer, each of the predicted molecular quantities based on the characteristics of the at least one monomer; generating, by the reaction module, a plurality of predicted representations of the complex amorphous polymer based on the characteristics of the at least one monomer; generating, by the reaction module, forcefield values for each of one or more molecules identified in each of the plurality of predicted representations; analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

Clause 16, the computing system of Clause 15, wherein the characteristics of the at least one monomer comprise at least one of: a simplified molecular-input line-entry system (SMILES) representation or a desired stoichiometric ratio relative to the reaction product model.

Clause 17, the computing system of Clause 15, further comprising calculating, by the reaction module, a molecular topological descriptor for each of the plurality of predicted representations.

Clause 18, the computing system of Clause 15, further comprising clustering, by the reaction module via the second trained machine learning model, each of the plurality of predicted representations into one or more groups, each of the one or more groups based on one or more structural similarities of each of the plurality of predicted representations.

Clause 19, the computing system of Clause 15, further comprising generating, by the reaction module, geometric values for each of the plurality of predicted representations.

Clause 20, the computing system of Clause 15, wherein each of the first trained machine learning model and the second trained machine learning model are selected from the group consisting of: a deep neural network, a decision tree algorithm, or a clustering algorithm.

Clause 21, the computing system of Clause 15, wherein each of the plurality of predicted representations comprises a simplified molecular-input line-entry system (SMILES) representation of the complex amorphous polymer.

What is claimed is:

1. A computer-implemented method for simulating a complex amorphous polymer using a reaction module and a processor, the method comprising:
receiving, by the processor, user input representative of characteristics of at least one monomer;
producing, by the processor via a first trained machine learning model, a predicted molecular quantity for each of the at least one monomer, each of the predicted molecular quantities based on the characteristics of the at least one monomer;
generating, by the reaction module, a plurality of predicted representations of the complex amorphous polymer based on the characteristics of the at least one monomer;
generating, by the reaction module, forcefield values for each of one or more molecules identified in each of the plurality of predicted representations;
analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and
assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

2. The method of claim 1, wherein the characteristics of the at least one monomer comprise at least one of: a simplified molecular-input line-entry system (SMILES) representation or a desired stoichiometric ratio relative to the reaction product model.

3. The method of claim 1, further comprising calculating, by the reaction module, a molecular topological descriptor for each of the plurality of predicted representations.

4. The method of claim 1, further comprising clustering, by the reaction module via the second trained machine learning model, each of the plurality of predicted representations into one or more groups, each of the one or more groups based on one or more structural similarities of each of the plurality of predicted representations.

5. The method of claim 1, further comprising generating, by the reaction module, geometric values for each of the plurality of predicted representations.

6. The method of claim 1, wherein each of the first trained machine learning model and the second trained machine learning model are selected from the group consisting of: a deep neural network, a decision tree algorithm, or a clustering algorithm.

7. The method of claim 1, wherein each of the plurality of predicted representations comprises a simplified molecular-input line-entry system (SMILES) representation of the complex amorphous polymer.

8. A computer program product for simulating a complex amorphous polymer, the computer program product comprising a computer readable storage device having program instructions embodied therewith, the program instructions executable by a processor to cause the processor to perform:
receiving, by a processor, user input representative of characteristics of at least one monomer;
producing, by the processor via a first trained machine learning model, a predicted molecular quantity for each of the at least one monomer, each of the predicted molecular quantities based on the characteristics of the at least one monomer;
generating, by a reaction module, a plurality of predicted representations of the complex amorphous polymer based on the characteristics of the at least one monomer;
generating, by the reaction module, forcefield values for each of one or more molecules identified in each of the plurality of predicted representations;
analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and
assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

9. The computer program product of claim 8, wherein the characteristics of the at least one monomer comprise at least one of: a simplified molecular-input line-entry system (SMILES) representation or a desired stoichiometric ratio relative to the reaction product model.

10. The computer program product of claim 8, further comprising calculating, by the reaction module, a molecular topological descriptor for each of the plurality of predicted representations.

11. The computer program product of claim 8, further comprising clustering, by the reaction module via the second trained machine learning model, each of the plurality of predicted representations into one or more groups, each of the one or more groups based on one or more structural similarities of each of the plurality of predicted representations.

12. The computer program product of claim 8, further comprising generating, by the reaction module, geometric values for each of the plurality of predicted representations.

13. The computer program product of claim 8, wherein each of the first trained machine learning model and the second trained machine learning model are selected from the group consisting of: a deep neural network, a decision tree algorithm, or a clustering algorithm.

14. The computer program product of claim 8, wherein each of the plurality of predicted representations comprises a simplified molecular-input line-entry system (SMILES) representation of the complex amorphous polymer.

15. A computing system comprising:
a processor,
a computer-readable storage device coupled to the processor;
a reaction module coupled to the processor; and
program instructions stored on the computer readable storage device for execution by the processor via a memory, wherein execution of the instructions by the processor configures the computing system to perform a complex amorphous polymer simulation method comprising:
receiving, by the processor, user input representative of characteristics of at least one monomer;
producing, by the processor via a first trained machine learning model, a predicted molecular quantity for each of the at least one monomer, each of the predicted molecular quantities based on the characteristics of the at least one monomer;
generating, by the reaction module, a plurality of predicted representations of the complex amorphous polymer based on the characteristics of the at least one monomer;
generating, by the reaction module, forcefield values for each of one or more molecules identified in each of the plurality of predicted representations;
analyzing, by the reaction module, a charge distribution of each of the plurality of predicted representations, wherein one or more surplus charges identified in one or more of the plurality of predicted representations are redistributed among one or more atoms of a respective one or more of the plurality of predicted representations; and
assembling, by the reaction module, a simulation box comprising a reaction product model including the plurality of predicted representations, wherein a quantity of each of the plurality of predicted representations in the simulation box is determined by weights of each of the plurality of predicted representations calculated by a second trained machine learning model.

16. The computing system of claim 15, wherein the characteristics of the at least one monomer comprise at least one of: a simplified molecular-input line-entry system (SMILES) representation or a desired stoichiometric ratio relative to the reaction product model.

17. The computing system of claim 15, further comprising calculating, by the reaction module, a molecular topological descriptor for each of the plurality of predicted representations.

18. The computing system of claim 15, further comprising clustering, by the reaction module via the second trained machine learning model, each of the plurality of predicted representations into one or more groups, each of the one or more groups based on one or more structural similarities of each of the plurality of predicted representations.

19. The computing system of claim 15, further comprising generating, by the reaction module, geometric values for each of the plurality of predicted representations.

20. The computing system of claim 15, wherein each of the first trained machine learning model and the second trained machine learning model are selected from the group consisting of: a deep neural network, a decision tree algorithm, or a clustering algorithm.

21. The computing system of claim 15, wherein each of the plurality of predicted representations comprises a simplified molecular-input line-entry system (SMILES) representation of the complex amorphous polymer.
